(19) United States
(12) Patent Application Publication
(10) Pub. No.: US 2025/0268279 A1
(43) Pub. Date: Aug. 28, 2025

(54) PREPARATION METHOD FOR LIGHT-COLORED WATER-SOLUBLE VEGETABLE PROTEIN

(71) Applicants: NANJING NUTRABUILDING BIO-TECH CO., LTD., Nanjing (CN); AXIOM FOODS, INC., Los Angeles, CA (US)

(72) Inventors: Kai ZHANG, Nanjing (CN); Jiasen YANG, Nanjing (CN); Qilin LIAO, Nanjing (CN)

(21) Appl. No.: 19/208,829

(22) Filed: May 15, 2025

Related U.S. Application Data
(63) Continuation of application No. PCT/CN2023/088567, filed on Apr. 17, 2023.

(30) Foreign Application Priority Data
Nov. 15, 2022 (CN) 202211424180.7

(51) Int. Cl. A23J 3/34; A23J 3/14; A23J 3/16; A23J 3/18; C07K 1/02; C07K 14/415

(52) U.S. Cl. CPC A23J 3/346; A23J 3/14; A23J 3/16; A23J 3/18; C07K 1/02; C07K 14/415

(57) ABSTRACT

The present disclosure provides a preparation method for a light-colored water-soluble vegetable protein. The method comprises: adding protease and glucose oxidase into a vegetable protein solution for enzymolysis to obtain a water-soluble vegetable protein. The method effectively reduces browning reactions in the enzymolysis process, not only lightening the color, but also ensuring that the yield of polypeptide products remains undiminished.

### CROSS REFERENCE TO RELATED APPLICATION

This application is a continuation application of International Application No. PCT/CN2023/088567, filed on Apr. 17, 2023, which is based upon and claims the priority to Chinese Patent Application No. 202211424180.7, filed on Nov. 15, 2022. The contents of International Application No. PCT/CN2023/088567 and Chinese Patent Application No. 202211424180.7 are incorporated herein by reference in their entireties.

### TECHNICAL FIELD

The present disclosure belongs to the technical field of vegetable protein deep processing, and particularly relates to a preparation method for light-colored water-soluble vegetable protein.

### BACKGROUND

As a renewable resource, vegetable protein or plant protein is another source of protein needed by human beings besides animal protein, which plays an important role in physiological regulation of human health. Compared with animal protein, vegetable protein production has stronger sustainability and lower environmental impact. Studies have shown that people who eat vegetable protein are significantly less likely to suffer from cardiovascular diseases than those who eat animal protein. Eating more vegetable protein can prolong the life of patients with chronic kidney disease and prevent diabetes. Many vegetable proteins have poor stability and poor absorption, which limits their application in food. The solubility of vegetable protein, especially cereal vegetable protein, in aqueous medium is very low, which seriously hinders the absorption and utilization of it by human body. Therefore, in order to improve the solubility of vegetable protein, it is necessary to reduce the molecular size of vegetable protein, promote the interaction between ionic protein and water, and weaken the interaction between hydrophobic protein.

Water-soluble vegetable protein is a kind of small molecular polypeptide formed by hydrolysis of vegetable protein under specific conditions. It is a kind of molecular polymer with a molecular weight of 100 to 10000 Da and a structure between amino acid and protein. It usually has 2 to 7 amino acid residues, and has low molecular weight, which is easy to be digested and absorbed by human body. At the same time, it has special physiological functions and has a certain regulatory effect on human life activities. Enzymatic hydrolysis of vegetable protein to prepare water-soluble vegetable protein refers to the method of using protein hydrolase to enzymatically hydrolyze vegetable protein as substrate to prepare water-soluble vegetable protein. Enzymatic hydrolysis has the advantages of mild reaction conditions, simple operation, easy control of hydrolysis conditions, less damage to protein and polypeptide nutrients, better absorption of peptides than proteins and amino acids, and generally better biological activity. However, vegetable protein contains a certain proportion of sugars, and after extracting and separating protein from it, some sugars will still remain in it. These sugars will have Maillard reaction with protein during enzymolysis to produce brown or even dark brown substances, which will deepen the color of the enzymatic hydrolysate. At present, the main measures to inhibit Maillard reaction include removing substances that promote browning reaction, adjusting influencing factors that promote browning reaction, and using browning inhibitors.

The problem of deep color in the preparation of water-soluble vegetable protein will greatly affect its commercial value. If decolorizing agent such as activated carbon is added subsequently to decolorize, more protein will be lost and the yield will be greatly reduced. Therefore, there is a need in the field to find a suitable method to improve the color deepening problem of water-soluble vegetable protein enzymatic hydrolysate.

### SUMMARY

GlucoseOxidase (E.C.1.1.3.4, GOD) is a very important oxidoreductase, which plays many important roles in biological processes. It can transform aldehyde groups on glucose molecules into carboxyl groups, generate gluconic acid and avoid browning. It is a natural food additive, which is non-toxic and has no side effects. It has been widely used in chemical industry, medicine, food, beverage, clinical chemistry, biotechnology and other industries. As a deglucose and deoxidizer, GlucoseOxidase has obvious effects in inhibiting browning of fruit juice and keeping fresh.

In order to solve the above problems, the present disclosure provides a method of adding glucose oxidase during enzymolysis, which effectively reduces the browning reaction in the enzymolysis process, not only lightens the color, but also ensures no loss in the polypeptide yield.

In the first aspect, the present disclosure provides a preparation method for a light-colored water-soluble vegetable protein, the method comprising: adding protease and glucose oxidase into a vegetable protein solution for enzymolysis to obtain the water-soluble vegetable protein.

In some embodiments, the method further comprises: mixing a vegetable protein with water in a ratio of material to liquid of 1:5 to 1:15 to obtain the vegetable protein solution.

In some embodiments, a sodium sulfite is added into the vegetable protein solution.

In some embodiments, the amount of the sodium sulfite is 0.001% to 0.1%, preferably 0.005% to 0.04% of that of the vegetable protein.

In some embodiments, the vegetable protein includes one or more of soybean protein, rapeseed protein, wheat protein, buckwheat protein, corn protein, barley protein, potato protein, rough rice protein, oat protein, pea protein, rice protein, pumpkin seed protein, watermelon seed protein, sunflower seed protein.

In some embodiments, the amount of the glucose oxidase is 0.02% to 5%, preferably 0.1% to 0.5% of that of the vegetable protein.

In some embodiments, the protease is alkaline protease and complex protease.

In some embodiments, the amount of the alkaline protease is 1% to 10%, preferably 1% to 3% of that of the vegetable protein, and the amount of the complex protease is 0.1% to 10%, preferably 0.2% to 2.5% of that of the vegetable protein.

In a second aspect, the present disclosure provides the water-soluble vegetable protein obtained by the method of the present disclosure, for use in preparing a food, beverage, nutrition, dietary supplement, cosmetic or medicine. The prepared food, beverage, nutrition, dietary supplement, cosmetic or medicine can be in the form of capsules, tablets, powders, suspensions, solutions, drops, granules, liquids, syrups, functional foods, beverages, toothpaste, sublingual products, food products, food additives, sweets, suckers, lozenges, food supplements, suppositories.

In a third aspect, the present disclosure provides use of glucose oxidase in improving color darkening of food, dietary supplement or nutrition comprising water-soluble vegetable protein.

The color of water-soluble vegetable protein is deep in the process of enzymatic hydrolysis preparation, and more protein will be lost when decoloring agents such as activated carbon are added later, which will reduce the yield. The method of the present disclosure solves the deep color problem from the source, in particular, adding glucose oxidase in the enzymolysis process can effectively inhibit browning, so that the problem of deep color in the preparation process of water-soluble vegetable protein is greatly improved. In addition, by using sodium sulfite to pretreat the vegetable protein solution, combining chemical reagents with enzymes, the browning problem in the enzymolysis process is better solved, and the finally prepared water-soluble vegetable protein is light in color and more acceptable in color, which is suitable for large-scale production and commercial application of water-soluble vegetable protein.

### DETAILED DESCRIPTION OF THE EMBODIMENTS

In the Summary Section above and the Detailed Description Section, and the claims below, reference is made to particular features of the present disclosure. It is to be understood that the disclosure of the present disclosure in this specification includes all possible combinations of such particular features. For example, where a particular feature is disclosed in the context of a particular aspect or embodiment of the present disclosure, or a particular claim, that feature can also be used, to the extent possible, in combination with and/or in the context of other particular aspects and embodiments of the present disclosure, and in the present disclosure generally.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, “light color” or “light-colored” refers to white, beige, milky white, ivory white, light yellow, light green, light pink, light blue, light purple and the like, which is suitable for products with color requirements. “Improving color darkening” means lightening the color; keeping light color; keeping it from browning; pigmenting or darkening; rendering the color light(er).

Enzymatic hydrolysis of vegetable protein to prepare water-soluble vegetable protein refers to the method of using protein hydrolase to enzymatically hydrolyze vegetable protein as substrate to prepare water-soluble vegetable protein.

Water-soluble vegetable protein is prepared by hydrolysis or enzymolysis of vegetable protein under specific conditions.

### EXAMPLES

In the present disclosure, unless otherwise specified, scientific and technical terms used herein have the meanings commonly understood by those skilled in the art. Moreover, the laboratory operation steps used herein are all routine steps widely used in the corresponding fields.

The raw materials or reagents used in the present disclosure are commercially available unless otherwise specified. All reagents are commercial grade and used according to the received standards.

The present disclosure provides a method for improving the color darkening in the enzymolysis preparation of water-soluble vegetable protein, which comprises the following steps: heating a vegetable protein solution, adjusting the pH value, and then adding alkaline protease, complex protease and glucose oxidase into the solution for enzymolysis reaction, thereby obtaining the water-soluble vegetable protein. The skilled person can determine the suitable enzymolysis temperature or pH or time, for example, the vegetable protein is hydrolyzed with enzyme at a pH between 4 and 9, a pH between 5 and 8.5, such as a pH between 6 and 8, e.g., a pH of about 8. The suitable temperature for the hydrolysis of vegetable protein with enzyme can be between 20° C. and 60° C., such as between 30° C. and 60° C., e.g., about 55° C. Specifically, for example, the vegetable protein solution can be heated to 55° C., the pH value can be adjusted to 8.0, and the enzymolysis reaction can be carried out for 4-6 hours, and the rotation speed of the enzymolysis reaction is 150-300 rpm, preferably 250 rpm. The addition amount of alkaline protease is 1% to 10%, preferably 1% to 3% of that of vegetable protein; the addition amount of complex protease is 0.1% to 10%, preferably 0.2% to 2.5% of that of vegetable protein; and the addition amount of glucose oxidase is 0.02% to 5%, preferably 0.1% to 0.5% of that of vegetable protein.

The vegetable protein of the present disclosure includes one or more of soybean protein, rapeseed protein, wheat protein, buckwheat protein, corn protein, barley protein, potato protein, rough rice protein, oat protein, pea protein, rice protein, pumpkin seed protein, watermelon seed protein, sunflower seed protein.

The vegetable protein solution in the above steps can be obtained by mixing vegetable protein with water in the ratio of material to liquid of 1:5 to 1:15. In addition, sodium sulfite can be added, and the addition amount of sodium sulfite is 0.001% to 0.1%, preferably 0.005% to 0.04% of that of vegetable protein.

In addition, the steps also include: after the enzymatic hydrolysis reaction is completed, increasing the temperature to inactivate the enzymes for 10-30 min, preferably 30 min, at 85-100° C., preferably 85° C. Filtering and collecting the filtrate; and filtering with a plate-and-frame filter, wherein a 3-5 μm filter membrane can be used in the plate-and-frame filter, and 0.5% to 3% perlite can be added to the enzymolysis liquid when filtering with the plate-and-frame filter. And then carrying out nanofiltration concentration, and spray-drying the nanofiltration concentrate, wherein the protein concentration after nanofiltration concentration is 14-16%, and the spray-drying conditions are that the air inlet temperature is 160-180°° C., and the air outlet temperature is 70-85° C., thereby obtaining water-soluble vegetable protein.

### Example 1

S1:3 kg of wheat protein with protein content over 80% was weighed into a bioreactor, 27 L of water was added, fully stirred and mixed, to obtain a mixed solution, which was proceeded to S2.

S2: The reactor was heated to 55° C. and the pH was adjusted to 8.0 with 4 M NaOH. Then 60 g of alkaline protease and 30 g of complex protease were added for enzymolysis. In the process of enzymolysis, the temperature in the reactor was kept at 55° C. and the rotation speed at 250 rpm. After 5 h of enzymolysis, it was proceeded to S3.

S3: The enzymatic hydrolysate was heated to 85° C. and kept for 30 min to inactivate the enzymes, and then proceeded to S4.

S4: The enzymatic hydrolysate was naturally cooled to 55° C., 300 g perlite was added to the enzymatic hydrolysate, stirred and mixed evenly, filtered by a plate-and-frame filter, and 25 L of filtrate was collected. The filtrate was concentrated by nanofiltration, and the protein content of the concentrated solution was 14.4%. Then it was proceeded to S5.

S5: The concentrated solution was spray dried at the air inlet temperature of 180° C. and the air outlet temperature of 75° C. to obtain water-soluble wheat protein.

The water-soluble wheat protein product contains 90.2% protein, 85% polypeptide, 82% peptide with molecular weight less than 1000 Da and 6.1% free amino acid. The water-soluble wheat protein was dissolved in water at a concentration of 5%, and the chromaticity was measured to be 1245 Hazen.

### Example 2

S1:3 kg of wheat protein with protein content over 80% was weighed into a bioreactor, 27 L of water was added, fully stirred and mixed, then 0.6 g of sodium sulfite was added, to obtain a mixed solution, which was proceeded to S2.

S2: The reactor was heated to 55° C. and the pH was adjusted to 8.0 with 4 M NaOH. Then 60 g of alkaline protease and 30 g of complex protease were added for enzymolysis. In the process of enzymolysis, the temperature in the reactor was kept at 55° C. and the rotation speed at 250 rpm. After 5 h of enzymolysis, it was proceeded to S3.

S3: The enzymatic hydrolysate was heated to 85° C. and kept for 30 min to inactivate the enzymes, and then proceeded to S4.

S4: The enzymatic hydrolysate was naturally cooled to 55° C., 300 g perlite was added to the enzymatic hydrolysate, stirred and mixed evenly, filtered by a plate-and-frame filter, and 25 L of filtrate was collected. The filtrate was concentrated by nanofiltration, and the protein content of the concentrated solution was 14.8%. Then it was proceeded to S5.

S5: The concentrated solution was spray dried at the air inlet temperature of 180° C. and the air outlet temperature of 75° C. to obtain water-soluble wheat protein.

The water-soluble wheat protein product contains 89% protein, 85.2% polypeptide, 84% peptide with molecular weight less than 1000 Da and 5.6% free amino acid. The water-soluble wheat protein was dissolved in water at a concentration of 5%, and the chromaticity was measured to be 932 Hazen.

### Example 3

S1:3 kg of wheat protein with protein content over 80% was weighed into a bioreactor, 27 L of water was added, fully stirred and mixed, to obtain a mixed solution, which was proceeded to S2.

S2: The reactor was heated to 55° C. and the pH was adjusted to 8.0 with 4 M NaOH. Then 60 g of alkaline protease, 30 g of complex protease, and 6 g of glucose oxidase were added for enzymolysis. In the process of enzymolysis, the temperature in the reactor was kept at 55° C. and the rotation speed at 250 rpm. After 5 h of enzymolysis, it was proceeded to S3.

S3: The enzymatic hydrolysate was heated to 85° C. and kept for 30 min to inactivate the enzymes, and then proceeded to S4.

S4: The enzymatic hydrolysate was naturally cooled to 55° C., 300 g perlite was added to the enzymatic hydrolysate, stirred and mixed evenly, filtered by a plate-and-frame filter, and 25 L of filtrate was collected. The filtrate was concentrated by nanofiltration, and the protein content of the concentrated solution was 14.5%. Then it was proceeded to S5.

S5: The concentrated solution was spray dried at the air inlet temperature of 180° C. and the air outlet temperature of 75° C. to obtain water-soluble wheat protein.

The water-soluble wheat protein product contains 88.5% protein, 84.4% polypeptide, 82.6% peptide with molecular weight less than 1000 Da and 6.3% free amino acid. The water-soluble wheat protein was dissolved in water at a concentration of 5%, and the chromaticity was measured to be 748 Hazen.

### Example 4

S1:3 kg of wheat protein with protein content over 80% was weighed into a bioreactor, 27 L of water was added, fully stirred and mixed, then 0.6 g of sodium sulfite was added, to obtain a mixed solution, which was proceeded to S2.

S2: The reactor was heated to 55° C. and the pH was adjusted to 8.0 with 4 M NaOH. Then 60 g of alkaline protease, 30 g of complex protease, and 6 g of glucose oxidase were added for enzymolysis. In the process of enzymolysis, the temperature in the reactor was kept at 55° C. and the rotation speed at 250 rpm. After 5 h of enzymolysis, it was proceeded to S3.

S3: The enzymatic hydrolysate was heated to 85° C. and kept for 30 min to inactivate the enzymes, and then proceeded to S4.

S4: The enzymatic hydrolysate was naturally cooled to 55° C., 300 g perlite was added to the enzymatic hydrolysate, stirred and mixed evenly, filtered by a plate-and-frame filter, and 25 L of filtrate was collected. The filtrate was concentrated by nanofiltration, and the protein content of the concentrated solution was 15.2%. Then it was proceeded to S5.

S5: The concentrated solution was spray dried at the air inlet temperature of 180° C. and the air outlet temperature of 75° C. to obtain water-soluble wheat protein.

The water-soluble wheat protein product contains 89% protein, 83.8% polypeptide, 84% peptide with molecular weight less than 1000 Da and 5.9% free amino acid. The water-soluble wheat protein was dissolved in water at a concentration of 5%, and the chromaticity was measured to be 681 Hazen.

### Example 5

S1:3 kg of rice protein with protein content over 80% was weighed into a bioreactor, 27 L of water was added, fully stirred and mixed, to obtain a mixed solution, which was proceeded to S2.

S2: The reactor was heated to 55° C. and the pH was adjusted to 8.0 with 4 M NaOH. Then 90 g of alkaline protease and 45 g of complex protease were added for enzymolysis. In the process of enzymolysis, the temperature in the reactor was kept at 55° C. and the rotation speed at 250 rpm. After 5 h of enzymolysis, it was proceeded to S3.

S3: The enzymatic hydrolysate was heated to 85° C. and kept for 30 min to inactivate the enzymes, and then proceeded to S4.

S4: The enzymatic hydrolysate was naturally cooled to 55° C., 600 g perlite was added to the enzymatic hydrolysate, stirred and mixed evenly, filtered by a plate-and-frame filter, and 24 L of filtrate was collected. The filtrate was concentrated by nanofiltration, and the protein content of the concentrated solution was 14.2%. Then it was proceeded to S5.

S5: The concentrated solution was spray dried at the air inlet temperature of 180° C. and the air outlet temperature of 75° C. to obtain water-soluble rice protein.

The water-soluble rice protein product contains 88.2% protein, 84.6% polypeptide, 83.5% peptide with molecular weight less than 1000 Da and 5.1% free amino acid. The water-soluble rice protein was dissolved in water at a concentration of 5%, and the chromaticity was measured to be 1052 Hazen.

### Example 6

S1:3 kg of rice protein with protein content over 85% was weighed into a bioreactor, 27 L of water was added, fully stirred and mixed, then 0.3 g of sodium sulfite was added, to obtain a mixed solution, which was proceeded to S2.

S2: The reactor was heated to 55° C. and the pH was adjusted to 8.0 with 4 M NaOH. Then 90 g of alkaline protease and 45 g of complex protease were added for enzymolysis. In the process of enzymolysis, the temperature in the reactor was kept at 55° C. and the rotation speed at 250 rpm. After 5 h of enzymolysis, it was proceeded to S3.

S3: The enzymatic hydrolysate was heated to 85° C. and kept for 30 min to inactivate the enzymes, and then proceeded to S4.

S4: The enzymatic hydrolysate was naturally cooled to 55° C., 600 g perlite was added to the enzymatic hydrolysate, stirred and mixed evenly, filtered by a plate-and-frame filter, and 24 L of filtrate was collected. The filtrate was concentrated by nanofiltration, and the protein content of the concentrated solution was 15.5%. Then it was proceeded to S5.

S5: The concentrated solution was spray dried at the air inlet temperature of 180° C. and the air outlet temperature of 75° C. to obtain water-soluble rice protein.

The water-soluble rice protein product contains 89.3% protein, 84.5% polypeptide, 84.2% peptide with molecular weight less than 1000 Da and 5.4% free amino acid. The water-soluble rice protein was dissolved in water at a concentration of 5%, and the chromaticity was measured to be 796 Hazen.

### Example 7

S1:3 kg of rice protein with protein content over 80% was weighed into a bioreactor, 27 L of water was added, fully stirred and mixed, to obtain a mixed solution, which was proceeded to S2.

S2: The reactor was heated to 55° C. and the pH was adjusted to 8.0 with 4 M NaOH. Then 90 g of alkaline protease, 45 g of complex protease, and 3 g of glucose oxidase were added for enzymolysis. In the process of enzymolysis, the temperature in the reactor was kept at 55° C. and the rotation speed at 250 rpm. After 5 h of enzymolysis, it was proceeded to S3.

S3: The enzymatic hydrolysate was heated to 85° C. and kept for 30 min to inactivate the enzymes, and then proceeded to S4.

S4: The enzymatic hydrolysate was naturally cooled to 55° C., 600 g perlite was added to the enzymatic hydrolysate, stirred and mixed evenly, filtered by a plate-and-frame filter, and 24 L of filtrate was collected. The filtrate was concentrated by nanofiltration, and the protein content of the concentrated solution was 14.1%. Then it was proceeded to S5.

S5: The concentrated solution was spray dried at the air inlet temperature of 180° C. and the air outlet temperature of 75° C. to obtain water-soluble rice protein.

The water-soluble rice protein product contains 90.2% protein, 83.6% polypeptide, 86.2% peptide with molecular weight less than 1000 Da and 6.6% free amino acid. The water-soluble rice protein was dissolved in water at a concentration of 5%, and the chromaticity was measured to be 670 Hazen.

### Example 8

S1:3 kg of rice protein with protein content over 85% was weighed into a bioreactor, 27 L of water was added, fully stirred and mixed, then 0.3 g of sodium sulfite was added, to obtain a mixed solution, which was proceeded to S2.

S2: The reactor was heated to 55° C. and the pH was adjusted to 8.0 with 4 M NaOH. Then 90 g of alkaline protease, 45 g of complex protease, and 3 g of glucose oxidase were added for enzymolysis. In the process of enzymolysis, the temperature in the reactor was kept at 55° C. and the rotation speed at 250 rpm. After 5 h of enzymolysis, it was proceeded to S3.

S3: The enzymatic hydrolysate was heated to 85° C. and kept for 30 min to inactivate the enzymes, and then proceeded to S4.

S4: The enzymatic hydrolysate was naturally cooled to 55° C., 600 g perlite was added to the enzymatic hydrolysate, stirred and mixed evenly, filtered by a plate-and-frame filter, and 24 L of filtrate was collected. The filtrate was concentrated by nanofiltration, and the protein content of the concentrated solution was 14.6%. Then it was proceeded to S5.

S5: The concentrated solution was spray dried at the air inlet temperature of 180° C. and the air outlet temperature of 75° C. to obtain water-soluble rice protein.

The water-soluble rice protein product contains 89.5% protein, 83.8% polypeptide, 85.6% peptide with molecular weight less than 1000 Da and 4.3% free amino acid. The water-soluble rice protein was dissolved in water at a concentration of 5%, and the chromaticity was measured to be 625 Hazen.

### Example 9

The vegetable protein of Examples 3 to 4 or Examples 7 to 8 was replaced by one or more of soybean protein, rapeseed protein, wheat protein, buckwheat protein, corn protein, barley protein, potato protein, rough rice protein, oat protein, pea protein, rice protein, pumpkin seed protein, watermelon seed protein, sunflower seed protein. And the appropriate reaction conditions were adjusted to prepare the corresponding water-soluble vegetable protein. The chromaticity of the obtained water-soluble vegetable protein is 560 Hazen to 750 Hazen.

Although specific embodiments and examples of the present disclosure have been illustrated herein, it will be appreciated by those skilled in the art that any modifications and variations can be made without departing from the spirit of the present disclosure. The examples and illustrations above are not intended to limit the scope of the present disclosure. Any combination of embodiments of the present disclosure, along with any obvious extension or analogs, are within the scope of the present disclosure. Further, it is intended that the present disclosure encompass any arrangement, which is calculated to achieve that same purpose, and all such variations and modifications as fall within the scope of the appended claims.

What is claimed is:

1. A preparation method for a light-colored water-soluble vegetable protein, the method comprising: adding protease and glucose oxidase into a vegetable protein solution for enzymolysis to obtain the light-colored water-soluble vegetable protein.

2. The method of claim 1, further comprising: mixing a vegetable protein with water in a ratio of 1:5 to 1:15 to obtain the vegetable protein solution.

3. The method of claim 2, further comprising: adding a sodium sulfite into the vegetable protein solution.

4. The method of claim 3, wherein an amount of the sodium sulfite is 0.001% to 0.1% of that of the vegetable protein.

5. The method of claim 1 wherein the vegetable protein is one or more of soybean protein, rapeseed protein, wheat protein, buckwheat protein, corn protein, barley protein, potato protein, rough rice protein, oat protein, pea protein, rice protein, pumpkin seed protein, watermelon seed protein, sunflower seed protein.

6. The method of claim 1, wherein an amount of the glucose oxidase is 0.02% to 5% of that of the vegetable protein.

7. The method of claim 1, wherein the protease is alkaline protease and complex protease.

8. The method of claim 7, wherein an amount of the alkaline protease is 1% to 10% of that of the vegetable protein, and an amount of the complex protease is 0.1% to 10% of that of the vegetable protein.

9. The light-colored water-soluble vegetable protein obtained by the method of claim 1, for use in preparing a food, beverage, nutrition, dietary supplement, cosmetic or medicine.

10. The light-colored water-soluble vegetable protein of claim 9, wherein the food, beverage, nutrition, dietary supplement, cosmetic or medicine is in a form of at least one of capsules, tablets, powders, suspensions, solutions, drops, granules, liquids, syrups, functional foods, beverages, toothpaste, sublingual products, food products, food additives, sweets, suckers, lozenges, food supplements, or suppositories.

11. A method of improving color darkening of food, dietary supplement or nutrition comprising water-soluble vegetable protein, wherein the method comprises using glucose oxidase in a preparation of the water-soluble vegetable protein from a vegetable protein.

12. The method of claim 11, wherein an amount of the glucose oxidase is 0.02% to 5% of that of the vegetable protein.

13. The method of claim 11, wherein the vegetable protein is one or more of soybean protein, rapeseed protein, wheat protein, buckwheat protein, corn protein, barley protein, potato protein, rough rice protein, oat protein, pea protein, rice protein, pumpkin seed protein, watermelon seed protein, sunflower seed protein.

14. The method of claim 11, wherein a sodium sulfite is added into the vegetable protein.

15. The method of claim 14, wherein an amount of the sodium sulfite is 0.001% to 0.1% of that of the vegetable protein.
